(19) United States
(12) Patent Application Publication
(10) Pub. No.: US 2016/0157843 A1
(43) Pub. Date: Jun. 9, 2016

(54) TISSUE BAG AND METHOD OF MORCELLATING TISSUE

(71) Applicant: GYRUS MEDICAL LIMITED, Cardiff (GB)

(72) Inventors: James Alan DICKSON, Cardiff (GB); Kerry Simpson Briggs, Cardiff (GB); Chun Keat OOI, Galway (IE)

(21) Appl. No.: 14/952,053

(22) Filed: Nov. 25, 2015

(30) Foreign Application Priority Data
Dec. 4, 2014 (GB) 1421588.3

(51) Int. Cl. A61B 17/00; A61B 17/221; A61B 18/14

(52) U.S. Cl. CPC A61B 17/00234; A61B 18/1482; A61B 17/221; A61B 2017/00287; A61B 2018/00559

(57) ABSTRACT

An apparatus for morcellating tissue includes a flexible outer container (1), an electrically conductive mesh (7) disposed within the container, and one or more connections (8, 9) by which the mesh (7) can be energised to form at least a part of a bipolar electrode assembly capable of cutting tissue coming into contact therewith. The mesh (7) includes a first section (14, 17) constituting one or more active electrodes, a second section (15, 18) constituting one or more return electrodes, and one or more insulators (16) separating the first section from the second section. The apparatus includes an elongate shaft (2) from which the flexible outer container (1) can be deployed, and actuators for closing the mesh (7) around tissue to be morcellated, and for deploying and withdrawing the flexible outer container (1) with respect to the shaft (2).

### CROSS-REFERENCE TO RELATED APPLICATION

This application claims priority to United Kingdom Application No. 1421588.3, filed 4 Dec. 2014, the entire contents of which are incorporated herein by reference.

### TECHNICAL FIELD

Embodiments of the invention relate to a tissue bag for use in the morcellation of tissue, and to a method for morcellating tissue such as a female uterus.

### BACKGROUND TO THE INVENTION AND PRIOR ART

Due to the perceived risks associated with the “seeding” of cancerous tissue, the morcellation of tissue is often carried out in a tissue bag surrounding or containing the tissue. An example of such a tissue bag is given in U.S. Pat. No. 5,037,379. In the tissue bag of U.S. Pat. No. 5,037,379, a morcellating instrument is introduced into the bag in order to morcellate the tissue into smaller pieces before the bag is removed from the body of the patient.

In other prior art, U.S. Pat. No. 5,735,289 describes a tissue bag in which a mechanical mesh is constricted in order to mechanically morcellate tissue contained within the bag. The document describes an option of connecting wires leading to the mesh to an electrical power source to perform “heating” of the enclosure to “cauterize” the tissue, but does not envisage using the current to perform the electrosurgical cutting or vaporisation of the tissue. This prior art device will therefore be of limited use for morcellation, due to the limitations imposed by the need for the tissue to be morcellated to be relatively soft, and hence capable of being mechanically morcellated through the wire mesh. The mechanical mesh morcellator will find it difficult to deal with relatively harder tissue, such as fibroid tissue often found within the uterus.

### SUMMARY OF THE INVENTION

Embodiments of the present invention attempt to provide an improvement to this type of morcellation process. Accordingly, from one aspect there is provided apparatus for morcellating tissue comprising a flexible outer container; an electrically conductive mesh disposed within the container, the mesh including a first section constituting one or more active electrodes, a second section constituting one or more return electrodes, and one or more insulators separating the first section from the second section, the apparatus including one or more connections by which the mesh can be energised to form a bipolar electrode assembly capable of cutting tissue coming into contact therewith. In contrast to U.S. Pat. No. 5,735,289, the bipolar energisation of the mesh allows for all types of tissue, hard and soft, to be successfully morcellated.

In one arrangement, the tissue to be morcellated is placed on to the mesh, which is then activated with an RF tissue-cutting waveform. The weight of the tissue resting of the mesh is sufficient to cause the tissue to be morcellated, with morcellated tissue passing through the mesh and being contained below the mesh within the flexible outer container. In other arrangements, the apparatus includes an actuator for closing the mesh around tissue to be morcellated. In this way, the mesh can be constricted around the tissue to be morcellated, rather than relying on the weight of the tissue resting on the mesh to cause sufficient contact between the mesh and the tissue. Conveniently, the actuator comprises a drawstring arrangement.

In a preferred arrangement, the apparatus includes an elongate shaft from which the flexible outer container can be deployed. Conveniently, the apparatus includes a second actuator for deploying and withdrawing the flexible outer container with respect to the shaft. The shaft is typically manoeuvred within the body of the patient in order to locate the outer container over the tissue to be morcellated, or for the tissue to be morcellated to be placed within the container. The mesh can then be energised to perform tissue cutting, and the actuator used to close the mesh around the tissue to be morcellated. The mesh can either be energised first, with the mesh then being closed around tissue to be morcellated, or vice versa, with the mesh first being closed around tissue by the actuator, before the mesh is energised.

According to one convenient arrangement, the first section constituting one or more active electrodes and the second section constituting one or more return electrodes comprise different areas of the mesh. The mesh is typically drawn into a spherical or spheroid configuration, with a longitudinal and a transverse axis. In one configuration, the first section comprises one transverse section of the mesh, typically the right hand half of the sphere or spheroid, with the second section comprising the opposite transverse section, typically the left hand half. A series of insulators or an insulated section of the mesh divides the first and second sections of the mesh.

Alternatively, the first section comprises one longitudinal section of the mesh, with the second section comprising the opposite longitudinal section. Typically “proximal” is defined as the direction towards the area where the apparatus is introduced into the body of the patient, and “distal” is defined as the direction away from where the apparatus is introduced. Conveniently, the first section is constituted by a distal section of the mesh, while the second section is constituted by a proximal section of the mesh. A series of insulators or an insulated section of the mesh divides the proximal and distal sections of the mesh.

Typically, the mesh is constituted by a network of electrically conductive wires, the wires crossing one another transversely at a plurality of nodes. According to an alternative construction to those described in the previous paragraphs, the first section is conveniently constituted by a first set of wires, and the second section is constituted by wires running transverse to the first set of wires, the one or more insulators being situated at the plurality of nodes. In the above arrangement, the first section is typically longitudinally extending wires with the second section being wires running transversely to the longitudinally extending wires, or vice versa. In this way, a criss-cross pattern of active and return wires is established, with tissue cutting occurring when current flows between the active wires and the return wires.

Embodiments of the invention further reside in a system for morcellating tissue, the system comprising a generator for supplying an RF waveform capable of vaporising tissue, and an instrument, the instrument comprising a flexible outer container, an electrically conductive mesh disposed within the container, and one or more connections by which the mesh can be connected to the generator such that the generator supplies the RF waveform to the mesh such that the mesh forms at least a part of a bipolar electrode assembly which vaporises tissue coming into contact therewith, the remaining debris passing through the mesh to be constrained within the flexible outer container.

Further embodiments are also provided by an apparatus for morcellating tissue comprising: a flexible outer container; an electrically conductive mesh disposed within the container, the mesh including one or more active electrodes; at least one return electrode located on the apparatus spatially separated from the electrically conductive mesh; and one or more connections by which the mesh can be energised to form, in conjunction with the separate return electrode, a bipolar electrode assembly capable of cutting tissue coming into contact therewith. In these further embodiments a separate return electrode is provided somewhere else on the instrument separate from the mesh, all of which (or at least a portion (and preferably a major portion) of which) constitutes the active electrode. This means that the mesh can be of simpler, and hence lower cost, construction, as there is no need for the mesh to include insulating elements to separate the active parts from the return parts.

Conveniently, where the apparatus includes an elongate shaft, the return electrode is typically located on the elongate shaft, either as a separate element or constituted by the shaft itself. Conceivably, the return electrode could be a portion of the flexible outer container. Whichever construction is employed, the bipolar electrode assembly established by the active and return electrodes allows for tissue contacting the mesh to be electrosurgically morcellated.

Embodiments of the invention further reside in a method of morcellating tissue within the body of a patient comprising the steps of

(i) introducing a tissue bag into the body of the patient, the tissue bag comprising a flexible outer container and an electrically conductive mesh disposed within the container,
(ii) placing the tissue to be morcellated into the tissue bag,
(iii) closing the tissue bag around the tissue to be morcellated,
(iv) energising the mesh with an RF waveform capable of cutting tissue coming into contact therewith,
(v) closing the mesh around the tissue to be morcellated in order to morcellate the tissue into small pieces, and,
(vi) removing the tissue bag containing the morcellated tissue from the body of the patient.

In the above method, tissue is placed into the tissue bag, having been previously mobilised or severed from surrounding tissue. Alternatively, embodiments of the invention also reside in a method of morcellating tissue within the body of a patient comprising the steps of

(i) introducing a tissue bag into the body of the patient, the tissue bag comprising a flexible outer container and an electrically conductive mesh disposed within the container,
(ii) placing the tissue bag over the tissue to be morcellated,
(iii) closing the tissue bag around the tissue to be morcellated,
(iv) energising the mesh with an RF waveform capable of cutting tissue coming into contact therewith,
(v) closing the mesh around the tissue to be morcellated in order to morcellate the tissue into small pieces, and,
(vi) removing the tissue bag containing the morcellated tissue from the body of the patient.

In this arrangement, the tissue bag is placed over the tissue to be morcellated, as opposed to the tissue being placed within the bag.

Embodiments of the present invention are particularly suited to the amputation and morcellation of a female uterus as part of a laparoscopic hysterectomy procedure (typically an LSH or LASH procedure). Accordingly, another embodiment of the invention resides in a method of amputating and morcellating a uterus comprising the steps of

(i) introducing a tissue bag into the abdomen of the patient, the tissue bag comprising a flexible outer container and an electrically conductive mesh disposed within the container,
(ii) placing the tissue bag over the uterus,
(iii) introducing a laparoscopic surgical instrument into the first tissue bag,
(iv) amputating the uterus using the laparoscopic surgical instrument,
(v) closing the first tissue bag around the amputated uterus,
(vi) energising the mesh with an RF waveform capable of cutting tissue coming into contact therewith,
(vii) closing the mesh around the uterus in order to morcellate the uterus into small pieces, and,
(viii) removing the tissue bag containing the morcellated uterus from the abdomen of the patient.

### DESCRIPTION FO THE DRAWINGS

Embodiments of the invention will now be further described, by way of example only, with reference to the accompanying drawings, in which

FIG. 1 is a perspective view of a tissue bag in accordance with an embodiment of the present invention,

FIGS. 2 to 4 are schematic sectional views showing the tissue bag of FIG. 1 being placed over a female uterus,

FIG. 5 is a schematic sectional view showing the tissue bag of FIG. 1 in accordance with a first embodiment of the invention,

FIG. 6 is a schematic sectional view showing the tissue bag of FIG. 1 in accordance with an alternative embodiment of the invention,

FIG. 7 is a schematic sectional view showing the tissue bag of FIG. 1 in accordance with a further embodiment of the invention,

FIG. 8 is a schematic sectional view showing the tissue bag of FIG. 1 in accordance with yet another embodiment of the invention,

FIGS. 9 & 10 are schematic sectional views showing tissue being removed from the tissue bag of FIG. 1, and

FIG. 11 is a schematic sectional view showing the tissue bag of FIG. 1 being removed from a patient.

### DESCRIPTION FO THE EMBODIMENTS

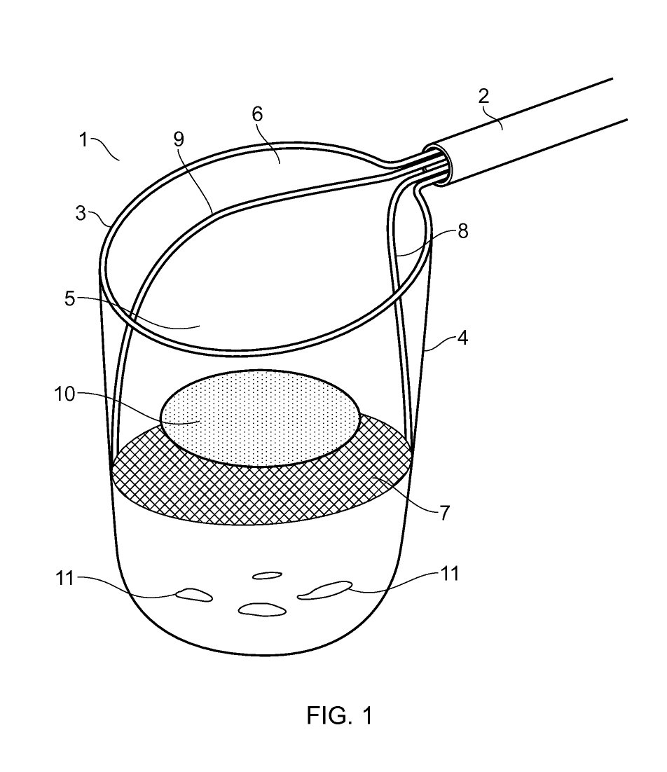

Referring to FIG. 1, a polyurethane tissue bag is shown generally at 1, deployed from an elongate shaft 2. The tissue bag 1 comprises a flexible ring 3 from which depends a transparent wall 4 forming an internal enclosure 5 with an opening 6 allowing access to the enclosure 5. The ring 3 ensures that the bag 1 assumes its expanded shape once it has been deployed from the shaft 2 within the body of a patient.

Within the enclosure 5 is a mesh 7 formed of metallic wires, constructed so as to form a bipolar tissue-cutting mechanism, as will be described in more detail subsequently. Leads 8 & 9 lead to the mesh 7, in order to supply an RF current to the bipolar mechanism, and also so as to act as a drawstring for closing the mesh around tissue, if required.

In use the shaft 2 is introduced into the body of a patient to be treated, and the tissue bag 1 is deployed from the shaft and kept open by means of a source of insufflation (not shown). Tissue to be morcellated such as a uterus 10 is placed into the enclosure 5 (by laparoscopic instruments—also not shown), so that it rests on the mesh 7. The mesh is then energised to activate the bipolar mechanism, causing tissue in contact with the mesh 7 to be morcellated into small pieces, which then drop through the mesh to rest at the bottom of the tissue bag as shown at 11. As the original tissue is to morcellated, further tissue comes in contact with the mesh and is morcellated accordingly. When sufficient tissue has been morcellated to reduce the size of the uterus 10 to a manageable size, the tissue bag 1 is withdrawn through the shaft 2, taking with it the morcellated tissue 11.

If the tissue is too large to be removed in this way, the mesh 7 can be removed through the shaft 2 as a first step. Then, with the tissue bag still maintained open by insufflation, large pieces of tissue can be removed by means of graspers (not shown) before the tissue bag 1 (together with any smaller pieces of tissue remaining therein) is removed through the shaft 2.

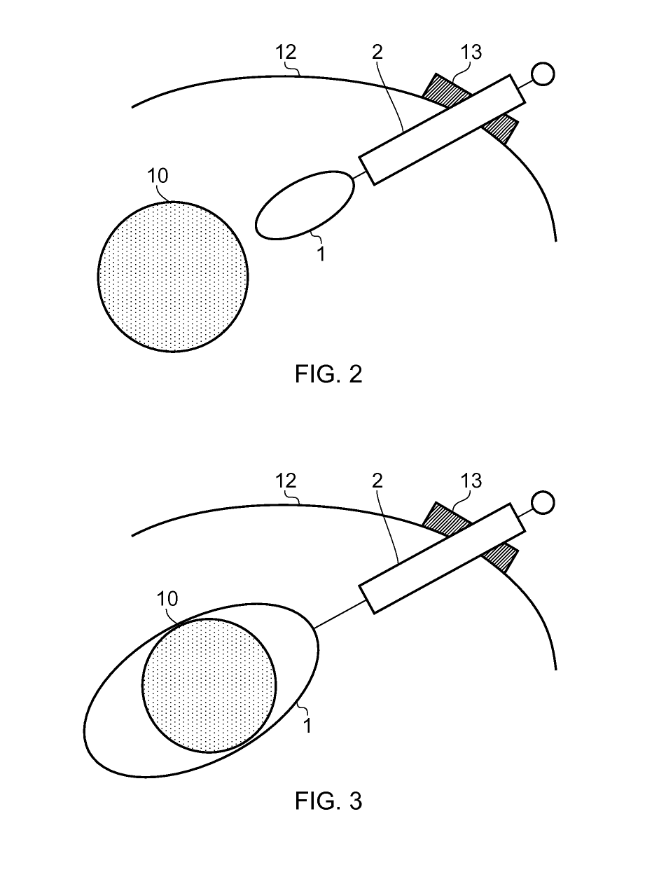

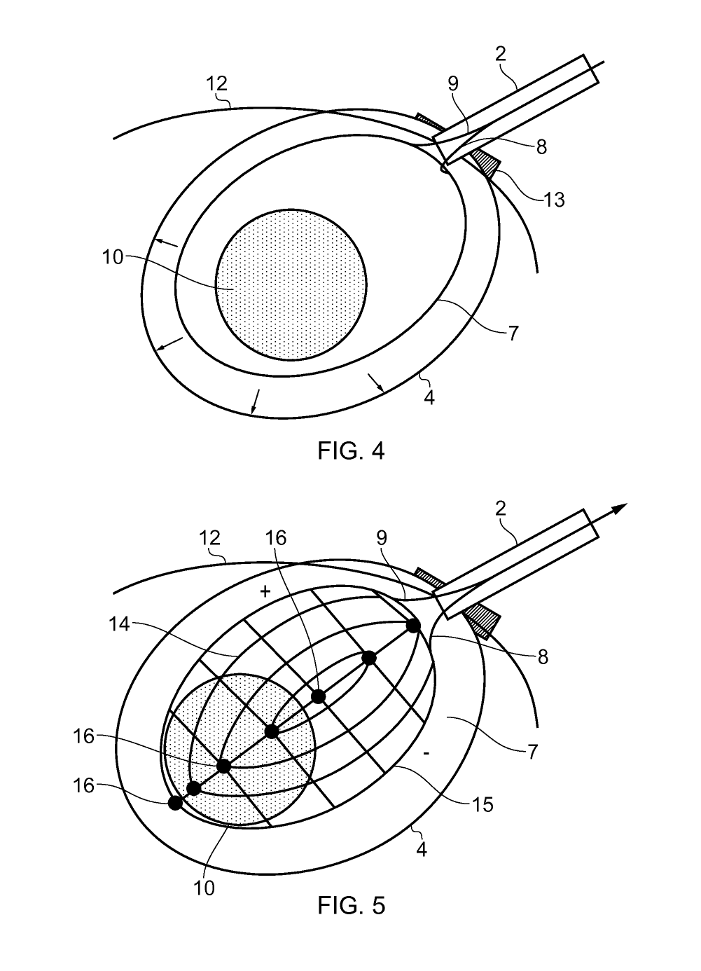

FIGS. 2 to 4 show the deployment of the tissue bag in more detail. In FIG. 2, the shaft 2 is introduced through the abdominal wall 12 of a patient through a port 13. The tissue bag 1 is deployed from the shaft and placed over the uterus 10 as shown in FIG. 3. The uterus is mobilised by severing it from the cervix, and the tissue bag 1 containing the uterus is manipulated so that the tissue bag is adjacent the port 13. The tissue bag 1 is then insufflated so that the mesh 7 is separated from the wall 4 of the tissue bag, and the leads 8 & 9 are withdrawn to close the mesh 7 around the uterus 10. This is the position shown in FIG. 4.

FIG. 5 shows one arrangement for the bipolar tissue-cutting mechanism. The mesh 7 is provided with a right hand section of wires 14, connected to the lead 9, and a left hand section of wires 15, connected to the lead 8. Between the sections 14 & 15 there are a plurality of insulators 16, electrically insulating the wires 14 from the wires 15. Leads 8 & 9 are connected to an electrosurgical generator (not shown), which supplies an electrosurgical cutting voltage between the leads 8 & 9 and hence between the sections 14 & 15. Tissue in the region of the insulators 16 is vaporised by the electrosurgical current passing between sections 14 & 15, causing the uterus 10 to be morcellated into small pieces, which pass through the mesh 7 into the tissue bag below.

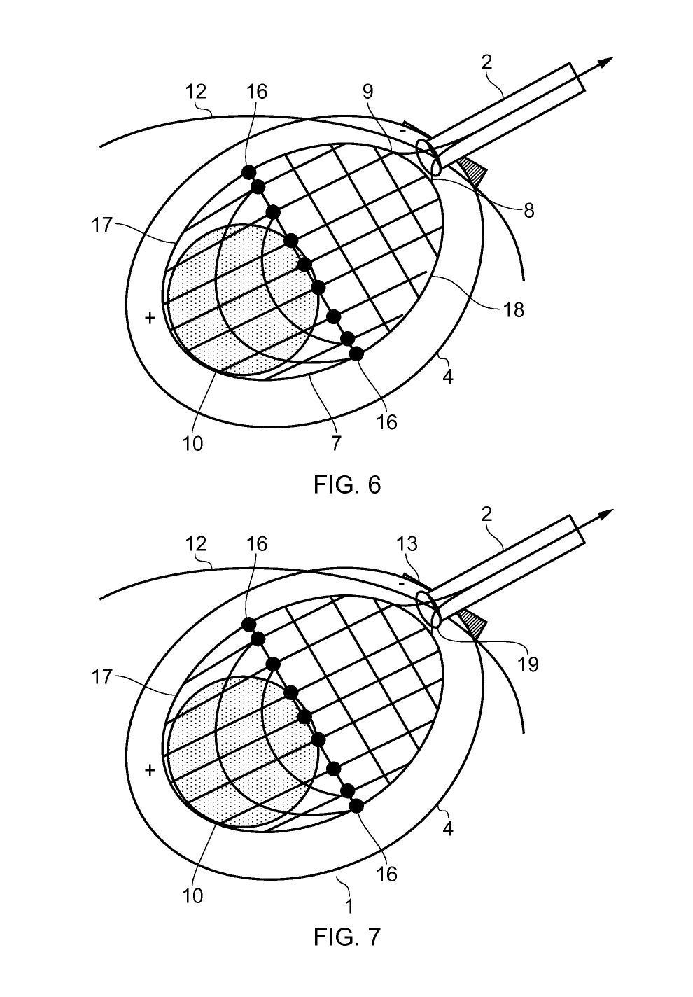

FIG. 6 shows an alternative arrangement in which the mesh 7 is provided with a distal section of wires 17 connected to the lead 9, and a proximal section of wires 18 connected to the lead 8. As before, insulators 16 separate and electrically insulate the wires in the distal section 17 from those in the proximal section 18. The electrosurgical cutting voltage supplied between the leads 8 & 9 and hence the sections 17 & 18 once again causes the tissue to be morcellated into small pieces, which pass through the mesh 7 into the tissue bag below.

FIG. 7 shows a variation on the arrangement of FIG. 6, in which the distal section 17 again forms the active electrode of the bipolar tissue-cutting arrangement. However, in FIG. 7, the return electrode is constituted not by the proximal section of the wires, but by a separate return electrode. The return electrode is in the form of a ring 19 carried by the shaft 2. As before, as the leads 8 & 9 are withdrawn, causing the mesh 7 to close around the uterus 10, the mesh starts to be received within the shaft 2. When the wires of the distal section 17 approach the return ring 19, a bipolar tissue-cutting mechanism is created due to the electrosurgical cutting voltage supplied between the wires 17 and the return ring 19. This causes tissue being pulled towards the shaft 2 to be morcellated into small pieces, for collection and later removal in the tissue bag 1.

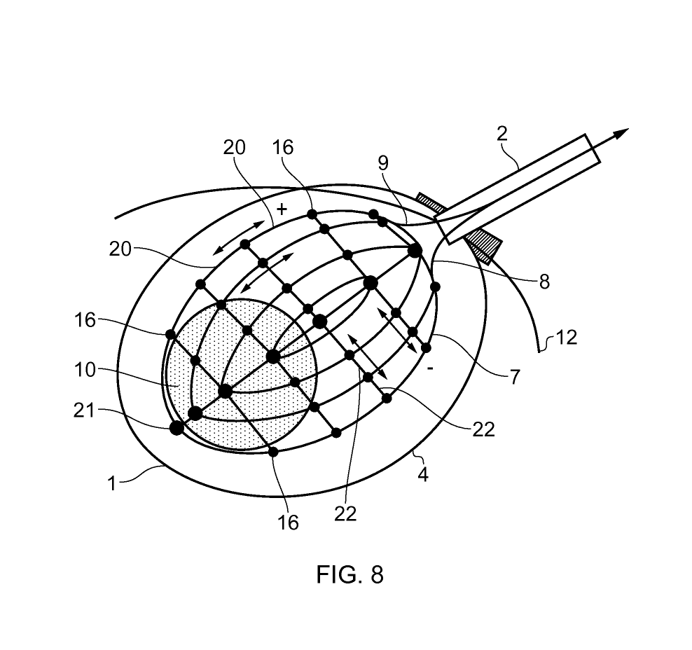

FIG. 8 shows a further alternative arrangement for the bipolar tissue-cutting mechanism, in which all the wires 20 running longitudinally from shaft 2 to the distal tip 21 of the spheroid mesh are connected in common to the lead 9. Similarly, all of the wires 22 running transversely around the mesh are connected in common to the lead 8. Insulators 16 are present at each and every node where the longitudinal wires meet the transverse wires. In this way, when the electrosurgical cutting voltage is supplied between leads 8 & 9 and hence the longitudinal wires 20 and the transverse wires 22, bipolar tissue-cutting pockets will be established with current passing between the longitudinal wires and the transverse wires via the tissue. Thus, tissue in contact with the mesh 7 will be vaporised, causing the uterus 10 to be quickly morcellated into small pieces, which pass through the mesh 7 and are collected in the tissue bag 1.

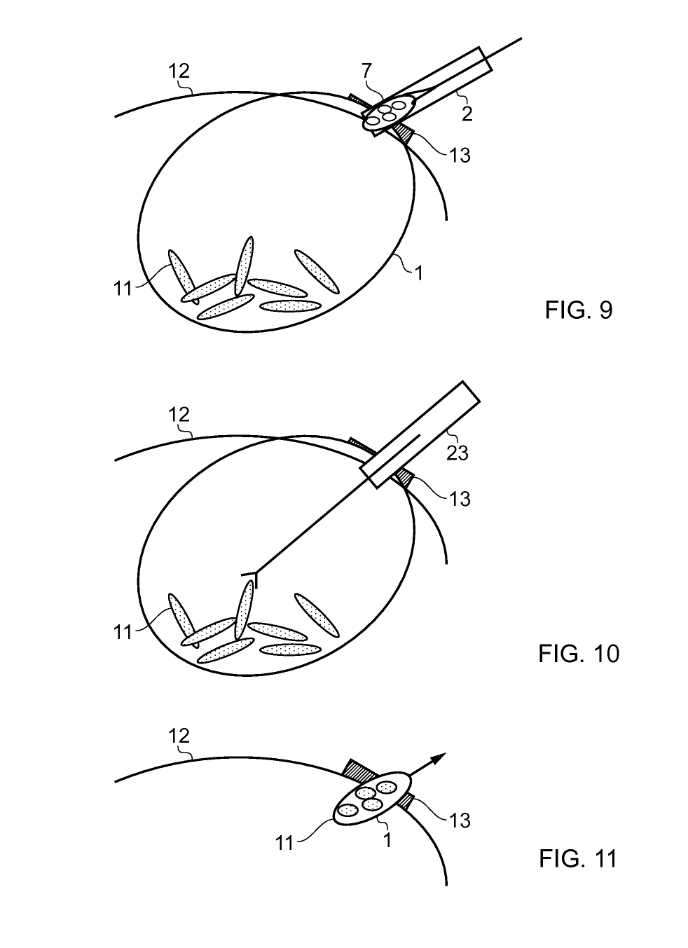

Whichever of the various bipolar combinations is employed, the uterus 10 will be morcellated by the electrosurgical cutting voltage as the mesh 7 is withdrawn into the shaft 2. FIG. 9 shows the mesh 7, together with small pieces of uterus contained therein, being received within the shaft 2. Pieces of morcellated tissue 11 having passed through the mesh 7 are constrained within the tissue bag 1. The larger of these pieces can be removed individually from the tissue bag using graspers 23 as shown in FIG. 10, before the tissue bag 1 and any remaining small pieces of morcellated tissue 11 is itself removed through the port 13 as shown in FIG. 11. In this way, the whole of the uterus 10 is morcellated and removed from the patient through the port 13, without the use of an additional morcellation instrument as required by prior art systems such as those of U.S. Pat. No. 5,735,289.

1. An apparatus for morcellating tissue comprising:
a flexible outer container;
an electrically conductive mesh disposed within the container, the mesh including a first section constituting one or more active electrodes, a second section constituting one or more return electrodes, and one or more insulators separating the first section from the second section, the apparatus including
one or more connections by which the mesh can be energised to form a bipolar electrode assembly capable of cutting tissue coming into contact therewith.

2. The apparatus according to claim 1, wherein the apparatus includes an actuator for closing the mesh around tissue to be morcellated.

3. The apparatus according to claim 2, wherein the actuator comprises a drawstring arrangement.

4. The apparatus according to claim 1, wherein the apparatus includes an elongate shaft from which the flexible outer container can be deployed.

5. The apparatus according to claim 4, wherein the apparatus includes a second actuator for deploying and withdrawing the flexible outer container with respect to the shaft.

6. The apparatus according to claim 1, wherein the first and second sections comprise different areas of the mesh.

7. The apparatus according to claim 6, wherein the mesh is constituted by a network of electrically conductive wires, the wires crossing one another transversely at a plurality of nodes.

8. The apparatus according to claim 7, wherein the first section is constituted by a first set of wires, and the second section is constituted by wires running transverse to the first set of wires, the one or more insulators being situated at the plurality of nodes.

9. A system for morcellating tissue, the system comprising:
a) a generator for supplying an RF waveform capable of vaporising tissue, and
b) an instrument, the instrument comprising
a flexible outer container;
an electrically conductive mesh disposed within the container, and
one or more connections by which the mesh can be connected to the generator such that the generator supplies the RF waveform to the mesh such that the mesh forms at least a part of a bipolar electrode assembly which vaporises tissue coming into contact therewith, the remaining debris passing through the mesh to be constrained within the flexible outer container.

10. A system according to claim 9, wherein the mesh includes a first section constituting one or more active electrodes.

11. A system according to claim 10, wherein the mesh also includes a second section constituting one or more return electrodes, and one or more insulators separating the first section from the second section.

12. A system according to claim 11, wherein the first and second sections comprise different areas of the mesh.

13. A system according to claim 9 wherein the instrument includes a return electrode separate from the mesh.

14. A system according to claim 13, wherein the return electrode comprises a portion of the flexible outer container.

15. A system according to claim 9, wherein the apparatus includes an elongate shaft from which the flexible outer container can be deployed.

16. A system according to claim 13, wherein the return electrode is located on the elongate shaft.

17. An apparatus for morcellating tissue comprising:
a flexible outer container;
an electrically conductive mesh disposed within the container, the mesh including one or more active electrodes;
at least one return electrode located on the apparatus spatially separated from the electrically conductive mesh; and
one or more connections by which the mesh can be energised to form, in conjunction with the separate return electrode, a bipolar electrode assembly capable of cutting tissue coming into contact therewith.

18. The apparatus according to claim 17, wherein the apparatus includes an actuator for closing the mesh around tissue to be morcellated.

19. The apparatus according to claim 18, wherein the actuator comprises a drawstring arrangement.

20. The apparatus according to claim 17, wherein the apparatus includes an elongate shaft from which the flexible outer container can be deployed.

21. The apparatus according to claim 20, wherein the apparatus includes a second actuator for deploying and withdrawing the flexible outer container with respect to the shaft.

22. A method of morcellating tissue within the body of a patient, the method comprising:
(i) introducing a tissue bag into the body of the patient, the tissue bag comprising a flexible outer container and an electrically conductive mesh disposed within the container,
(ii) placing the tissue to be morcellated into the tissue bag,
(iii) closing the tissue bag around the tissue to be morcellated,
(iv) energising the mesh with an RF waveform capable of cutting tissue coming into contact therewith,
(v) closing the mesh around the tissue to be morcellated in order to morcellate the tissue into small pieces, and,
(vi) removing the tissue bag containing the morcellated tissue from the body of the patient.

23. A method of morcellating tissue within the body of a patient, the method comprising:
(i) introducing a tissue bag into the body of the patient, the tissue bag comprising a flexible outer container and an electrically conductive mesh disposed within the container,
(ii) placing the tissue bag over the tissue to be morcellated,
(iii) closing the tissue bag around the tissue to be morcellated,
(iv) energising the mesh with an RF waveform capable of cutting tissue coming into contact therewith,
(v) closing the mesh around the tissue to be morcellated in order to morcellate the tissue into small pieces, and,
(vi) removing the tissue bag containing the morcellated tissue from the body of the patient.

24. A method of amputating and morcellating a uterus, the method comprising:
(i) introducing a tissue bag into the abdomen of the patient, the tissue bag comprising a flexible outer container and an electrically conductive mesh disposed within the container,
(ii) placing the tissue bag over the uterus,
(iii) introducing a laparoscopic surgical instrument into the first tissue bag,
(iv) amputating the uterus using the laparoscopic surgical instrument,
(v) closing the first tissue bag around the amputated uterus,
(vi) energising the mesh with an RF waveform capable of cutting tissue coming into contact therewith,
(vii) closing the mesh around the uterus in order to morcellate the uterus into small pieces, and,
(viii) removing the tissue bag containing the morcellated uterus from the abdomen of the patient.
